(19) United States
(12) Patent Application Publication
(10) Pub. No.: US 2016/0251612 A1
(43) Pub. Date: Sep. 1, 2016

(54) CULTURE MEDIUM FOR ESTABLISHING NEUROEPITHELIAL STEM CELLS, METHOD FOR ESTABLISHING NEUROEPITHELIAL STEM CELLS, AND APPLICATIONS THEREOF

(71) Applicant: YUNNAN KEY LAB OF PRIMATE BIOMEDICINE RESEARCH, Kunming City, Yunnan (CN)

(72) Inventors: Tianqing LI, Yunnan (CN); Weizhi JI, Yunnan (CN); Bo LI, Yunnan (CN); Xiaoqing ZHU, Yunnan (CN)

(21) Appl. No.: 14/770,283

(22) PCT Filed: Mar. 25, 2014
(86) PCT No.: PCT/CN2014/074029

§ 371 Date: Aug. 25, 2015

(51) Int. Cl. C12N 5/00; C12N 5/0797

(52) U.S. Cl. CPC C12N 5/0018; C12N 5/0623; C12N 2501/115; C12N 2501/415; C12N 2501/727; C12N 2501/15

(57) ABSTRACT

The present invention discloses a culture medium for establishing neuroepithelial stem cells, a method for establishing neuroepithelial stem cells, and the applications thereof, wherein the culture medium comprises differentiation culture medium for inducing and differentiating pluripotent stem cells to elementary neuroepithelial stem cells as well as proliferation culture medium for amplifying the elementary neuroepithelial stem cells; and the pluripotent stem cells can be differentiated to the elementary neuroepithelial stem cells in the differentiation culture medium. While the elementary neuroepithelial stem cells have the functions of the neuroepithelial stem cells, they cannot be stably amplified in long term culture under the conventional culture conditions, however they are maintained the stability properties in long term culture in the proliferation culture medium. The proliferation culture medium can culture the elementary neuroepithelial stem cells to the neuroepithelial stem cells, which are maintained stable amplification and survival, and can be produced in large scale.

### TECHNICAL FIELD

The present invention relates to the field of cell biology, particularly to a culture medium for establishing neuroepithelial stem cells, a method for establishing neuroepithelial stem cells, and the applications thereof.

### BACKGROUND

Neural diseases are critical diseases endangering human health, most of which are caused by the fact that the neural cells are irreplaceably damaged. It is very difficult to cure them using conventional therapies, however it is possible to cure these diseases by restoration and regeneration of neural stem cells.

The neural stem cells mainly undergo two phases during the development of the brain, i.e., neuroepithelial stem cells and radial glial progenitor cells. The neuroepithelial stem cells are the earliest neural stem cells with stronger differentiation multipotency, and are capable of differentiating and growing to form the whole brain cells. Also, the neuroepithelial stem cells have very strong proliferation ability to produce highly enriched neurons. These properties show neuroepithelial stem cells have important scientific and clinical application values.

However, in the related art, in the conventional culture method of neural stem cells, the combination of growth factors bFGF and EGF are generally used. Such culture systems can only culture and grow the later-stage neural progenitor cells (the radial glial progenitor cells), but are incapable of achieving long term and stable culture of neuroepithelial stem cells. Furthermore, the cultured radial glial progenitor cells significantly vary in cell properties and differentiation potential over the culture time, and lose the differentiation capability of neural cells. Therefore, these shortcomings disenable the large-scale production of the stem cells and limit their application in clinical stem cell replacement therapy, disease mechanism and drug screening.

Above all, one technical problem to be solved in the art is to establish neuroepithelial stem cells which can be stably passaged in vitro culture.

### DISCLOSURE OF THE INVENTION

The objective of the present invention is to provide a culture medium for establishing neuroepithelial stem cells, a method for establishing neuroepithelial stem cells, and the application thereof, for solving the above problems.

In an embodiment of the present invention, a culture medium is provided for establishing neuroepithelial stem cells, comprising:
(i) a neurobasal culture medium;
(ii) a B-27 supplement;
(iii) an N-2 supplement;
(iv) bFGF;
(v) an agonist of a Wnt signaling pathway,
(vi) a GSK inhibitor;
(vii) an inhibitor of a TGF-β signaling pathway, and
(viii) either both an inhibitor of a Notch signaling pathway and an inhibitor of an ALK2/ALK3 signaling pathway, or leukemia inhibitory factor.

A culture medium for establishing neuroepithelial stem cells according to the present invention comprises a differentiation culture medium as well as a proliferation culture medium, wherein the differentiation culture medium contains multiple kinds of nutritional ingredients necessary for differentiating the pluripotent stem cells to elementary neuroepithelial stem cells, wherein bFGF can promote early-stage differentiation and proliferation of neural stem cells; activation of the Wnt signaling pathway (by the agonist of the Wnt signaling pathway) facilitates proliferation of the neural stem cells; inhibition of the TGF-β signaling pathway promotes direct differentiation from pluripotent stem cells toward neural cells; inhibition of the Notch signaling pathway can rapidly promote direct differentiation from pluripotent stem cells toward neural cells and from neural stem cells toward neurons; and inhibition of the ALK2 and ALK3 signaling pathway can inhibit the BMP4 signaling pathway and promote direct differentiation from pluripotent stem cells toward neural cells and from neural stem cells toward neurons.

Thus, just using the differentiation culture medium containing the above ingredients, the pluripotent stem cells can be differentiated into elementary neuroepithelial stem cells. Elementary neuroepithelial stem cells have the functions of the neuroepithelial stem cells, but cannot be stably amplified for long term under the conventional culture conditions; however, neuroepithelial stem cells can be stably cultured over a long term using the proliferation culture medium, wherein bFGF, the agonist of the Wnt signaling pathway (GSK3 inhibitor), the inhibitor of TGF-β signaling pathway contained in the proliferation culture medium, all have functions of making the elementary neuroepithelial stem cells maintain its properties and stable proliferation abilities. In addition, Leukemia Inhibitory Factor regulates cell proliferation and differentiation. Just using the proliferation culture medium, the elementary neuroepithelial stem cells can be cultured to neuroepithelial stem cells which maintain stability and amplification abilities and can be produced on a large scale.

In one embodiment of the differentiation culture medium, the agonist of the Wnt signaling pathway and the GSK3 inhibitor are both CHIR99021; the inhibitor of the TGF-β signaling pathway is SB431542; the inhibitor of the Notch signaling pathway is Compound E; and the inhibitor of ALK2 and ALK3 signaling pathways is LDN193189; and/or in the proliferation culture medium, the agonist of the Wnt signaling pathway and the GSK3 inhibitor are both CHIR99021; and the inhibitor of TGF-β signaling pathway is SB4312542.

Structures of CHIR99021, SB431542, Compound E and LD193189 are all known, but are set forth here.

CHIR 99021 has the following chimerical structure:

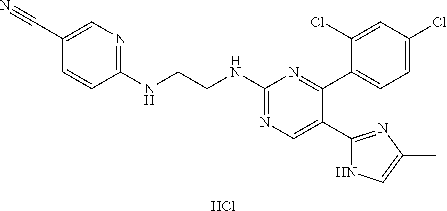 [SMILES: Cc1c[nH]c(-c2cnc(NCCNc3ccc(C#N)cn3)nc2-c2ccc(Cl)cc2Cl)n1 ; Cl]

SB431542 has the following chimerical structure:

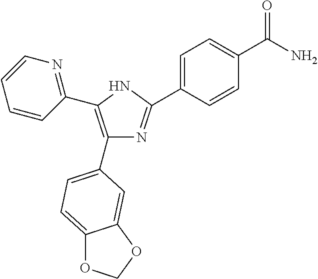 [SMILES: NC(=O)c1ccc(-c2nc(-c3ccc4c(c3)OCO4)c(-c3ccccn3)[nH]2)cc1]

LDN193189 has the following chimerical structure:

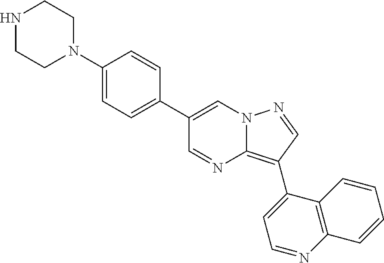 [SMILES: c1ccc2c(-c3cnn4cc(-c5ccc(N6CCNCC6)cc5)cnc34)ccnc2c1]

And Compound E has chimerical structure

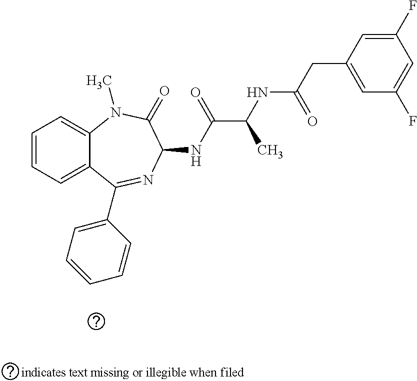 [SMILES: C[C@H](NC(=O)Cc1cc(F)cc(F)c1)C(=O)N[C@H]1N=C(c2ccccc2)c2ccccc2N(C)C1=O]

Optionally, in the differentiation culture medium, the concentration of the bFGF is 3-100 ng/ml; the concentration of the CHIR99021 is 0.3-30 μm/l, the concentration of the SB431542 is 2-50 μm/l; the concentration of the Compound E is 0.05-10 μm/l; and the concentration of the LDN193189 is 0.1-1 μm/l; and/or in the proliferation culture medium, the concentration of the bFGF is 3-100 ng/ml; the concentration of the CHIR99021 is 0.3-30 μm/l; the concentration of the SB431542 is 5-50 μm/l; the concentration of the Leukemia inhibitory factor is 50-5000 U/L.

Optionally, after the step of culturing the elementary neuroepithelial stem cells using the proliferation culture medium to obtain the neuroepithelial stem cells which can have stable proliferation ability, the method further comprises: culturing the neuroepithelial stem cells in neuron differentiation culture medium containing Neurobasal culture medium, B27, non-essential amino acids and glutamine, and differentiating them to obtain neurons with a purity of 40%-100%.

Optionally, the step of digesting the pluripotent stem cells with collagenase taken is 5-40 minutes.

Optionally, after the step of culturing the elementary neuroepithelial stem cells using the proliferation culture medium to obtain the neuroepithelial stem cells which can have stable proliferation ability, the method further comprises: digesting the neuroepithelial stem cells with 0.05% trypsin and being further passaged, to obtain a stable neuroepithelial stem cell line.

Neuroepithelial stem cells established according to the above method and a cell line produced by culturing said neuroepithelial stem cells are also a part of the invention.

Applications of the neuroepithelial stem cells and the cell line in cell replacement therapy, disease mechanism and drug screening, with the cell line produced by culturing the neuroepithelial stem cells are also a part of the invention.

### BRIEF DESCRIPTION OF DRAWINGS

In order to more clearly describe embodiments of the present invention or technical solutions in the prior art, brief introduction will be simply made to the below figures used in the description of the embodiments or the prior art. The figures described below are of some embodiments of the present invention, but other embodiments will be clear to the skilled artisan, without paying inventive labor.

FIG. 1 is a schematic view of Embodiment 2 of the present invention in which pluripotent stem cells are directly induced and differentiated to neuroepithelial stem cells;

FIG. 2 is a schematic view of Embodiment 2 of the present invention in which the neuroepithelial stem cells are cultured long term and on a large scale, and the stem cells are maintained to self-organize into a neural tube structure and produce highly enriched neurons;

FIG. 3 is a schematic view of Embodiment 2 of the present invention in which a single neuroepithelial stem cell self-organizes into the neural tube structure;

FIG. 4 is a schematic mechanism view of Embodiment 2 of the present invention in which the neuroepithelial stem cells self-organize into the neural tube structure and is converted to radial glial progenitor cells; and

FIG. 5 is a schematic view of Embodiment 2 of the present invention in which a single neuroepithelial stem cell is used to model human neural tube disease, differentiate to cortical neural cells and perform neural replacement therapy.

### DETAILED DESCRIPTION OF EMBODIMENTS

In order to make the objects, objectives and advantages of the present invention clearer, the technical solutions of the present invention will be described more clearly and completely by the descriptions below. Based on the embodiments of the present invention, one skilled in the art obtain, without inventive labor, other embodiments, all of which are covered by the present invention.

### Example I

The present embodiment provides a culture medium for establishing neuroepithelial stem cells, comprising: differentiation culture medium for inducing and differentiating pluripotent stem cells to elementary neuroepithelial stem cells as well as proliferation culture medium for amplifying the elementary neuroepithelial stem cells;

The differentiation culture medium comprises: Neurobasal culture medium, a B-27 supplement (a proprietary supplement for use with neural cells), a N-2 supplement (a proprietary serum free supplement for use with neural cells), bFGF, as agonist of the Wnt signaling pathway, a GSK3 inhibitor, an inhibitor of the TGF-β signaling pathway, an inhibitor of the Notch signaling pathway, and an inhibitor of the ALK2 and ALK3 signaling pathways. The proliferation culture medium comprises: Neurobasal culture medium, a B-27 supplement, N-2 supplement, bFGF, an agonist of Wnt signaling pathway, a GSK3 inhibitor, Leukemia Inhibitory Factor, and/or an inhibitor of TGF-β signaling pathway, and.

Such culture medium for establishing neuroepithelial stem cells according to the present invention comprises a differentiation culture medium as well as a proliferation culture medium. The differentiation culture medium contains multiple kinds of nutritional ingredients necessary for differentiating the pluripotent stem cells to the elementary neuroepithelial stem cells, wherein the bFGF can promote the early-stage generation and proliferation of neural stem cells; activation of the Wnt signaling pathway (by the agonist of the Wnt signaling pathway) facilitates the proliferation of the neural stem cells; inhibition of the TGF-β signaling pathway promotes the direct differentiation from the pluripotent stem cells toward neural cells; inhibition of the Notch signaling pathway can rapidly promote direct differentiation of the pluripotent stem cells toward neural cells and of the neural stem cells neurons; and inhibition of the ALK2 and ALK3 signaling pathway inhibit the BMP4 signaling pathway and promote the direct differentiation from the pluripotent stem cells toward neural cells and from the neural stem cells toward neurons.

Thus, just using the differentiation culture medium containing the above ingredients, the pluripotent stem cells can be differentiated to elementary neuroepithelial stem cells; while elementary neuroepithelial stem cells have the functions of the neuroepithelial stem cells, but cannot be stably amplified in long term culture under conventional culture conditions; however, they are stably cultured in long term using the proliferation culture medium, wherein the bFGF, the agonist of the Wnt signaling pathway (GSK3 inhibitor), the inhibitor of TGF-β signaling pathway contained in the proliferation culture medium are present. All to make the elementary neuroepithelial stem cells maintain their properties and stable amplification abilities. In addition, the Leukemia Inhibitory Factor functions as a regulator of cell proliferation and differentiation. Just using the proliferation culture medium, the elementary neuroepithelial stem cells can be cultured to the neuroepithelial stem cells which are maintained stably and can be amplified and be produced on a large scale.

More preferably, in the differentiation culture medium of the present embodiment, the agonist of the Wnt signaling pathway and the GSK3 inhibitor are both CHIR99021; the inhibitor of the TGF-β signaling pathway is SB431542; the inhibitor of the Notch signaling pathway is Compound E; and the inhibitor of ALK2 and ALK3 signaling pathways is LDN193189.

In the proliferation culture medium, the agonist of the Wnt signaling pathway and the GSK3 inhibitor are both CHIR99021, and the inhibitor of TGF-β signaling pathway is SB4312542.

More particularly, in the differentiation culture medium, the concentration of the bFGF is 3-100 ng/ml; the concentration of the CHIR99021 is 0.3-30 μm/l; the concentration of the SB431542 is 2-50 μm/l; the concentration of the Compound E is 0.55-10 μm/l; and the concentration of the LDN193189 is 0.1-10 μm/l; and in the proliferation culture medium, the concentration of the bFGF is 3-100 ng/ml; the concentration of the CHIR99021 is 0.3-30 μm/l; the concentration of the SB431542 is 5-50 μm/l; and the concentration of the Leukemia inhibitory factor 50-5000 U/L.

Examples of the differentiation culture medium and the proliferation culture medium of the present embodiment are as follows. As for the differentiation culture medium,

### Example 1

Neurobasal culture medium, B-27 supplement, N-2 supplement, 3 ng/ml bFGF, 0.3 μm/l CHIR99021, 2 μm/l SB431542, 0.05 μm/l Compound E, and 0.1 μm/l LDN193189;

### Example 2

Neurobasal culture medium, B-27 supplement, N-2 supplement, 100 ng/ml bFGF, 30 μm/l CHIR99021, 50 μm/l SB431542, 10 μm/l Compound E, and 10 μm/l LDN193189;

### Example 3

Neurobasal culture medium, B-27 supplement, N-2 supplement, 10 ng/ml bFGF, 3 m/l CHIR99021, 5 μm/l SB431542, 0.2 μm/l Compound E, and 0.1 m/l LDN193189.

As for the proliferation culture medium:

### Example 1

Neurobasal culture medium, B-27 supplement, N-2 supplement, 3 ng/ml bFGF, 0.3 μm/l CHIR99021, 5 μm/l SB431542, and 50 U/L Leukemia Inhibitory Factor;

### Example 2

Neurobasal culture medium, B-27 supplement, N-2 supplement, 100 ng/ml bFGF, 30 μm/l CHIR99021, 50 μm/l SB431542, and 5000 U/L Leukemia Inhibitory Factor;

### Example 3

Neurobasal culture medium, B-27 addictive, N-2 addictive, 10 ng/ml bFGF, 3 μm/l CHIR99021, 5 μm/l SB431542, and 1000 U/L Leukemia inhibitory factor.

### Example 2

A method for obtaining neuroepithelial stem cells using the culture medium according to Claim 1 is now described:

1. Digesting the pluripotent stem cells with collagenase to.

In particular, in this step, the pluripotent stem cells cultured with or without feeder layer are digested with collagenase (for 5-40 minutes) to small stem cell clusters (50-100 cells). In addition, in the present embodiment, the pluripotent stem cells comprise embryonic stem cells and induced pluripotent stem cells.

2. suspending the cell masses in the differentiation culture medium for suspension culture, so as to differentiate them to elementary neuroepithelial stem cells.

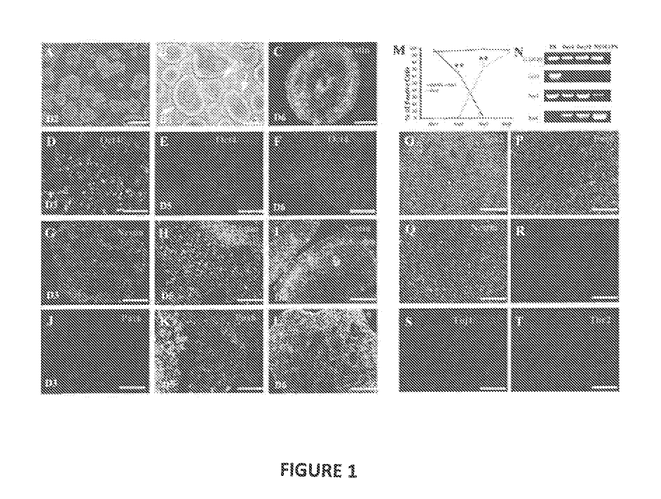

In particular, the cell clusters are suspended in the differentiation culture medium, cultured in suspension in a low-attachment culture dish, and differentiated for 6 days. The liquids are changed every two days. The 4th day post-differentiation, embryoid bodies formed two-layer neuroepithelial structures, shown in FIG. 1. The neuroepithelial structures of the 6th day post-differentiation are collected (by allowing cells to stand 5-10 minutes, and removing supernatant).

In FIG. 1, the procedure the pluripotent stem cells are directly induced and differentiated to the highly enriched neuroepithelial stem cells is shown, wherein (A) shows the typical embryoid bodies which are formed by the pluripotent stem cells in differentiation culture medium from the 2nd day post-differentiation; (B-C) show the neuroepithelial structures formed by the embryoid bodies, represented as Nestin, on the 5th to 6th day post-differentiation; (D-L) show positive cell staining results when the embryoid bodies (EBs) are cryosectioned and stained with Oct4, Sox2, Nestin and Pax6 on the 2nd, 5th and 6th day (D2, 5 and 6); (M) shows the quantitative change of the number of Sox2, Oct4 and Pax6 positive cells during the differentiation of the embryoid bodies, respectively (**P<0.01 representing significant change); (N) shows RT-PCR (mRNA reverse transcription PCR) of the embryoid bodies of the 6th day and 12th day post-differentiation and the amplified neuroepithelial stem cell samples of the 6th passage, which indicates that the pluripotent stem cells quickly lose pluripotency and are converted to neuroepithelial stem cells; and (O-T) show that the neuroepithelial stem cells cultured for long term express markers of stem cells, such as Sox2, Pax6, Nestin and N-cadherin, but not GFAP of the radial glial progenitor cells, the neuron protein Tuj1(s) and the progenitor cell marker protein Tbr2 of outer region of lateral ventricle in the developing cortex of human and monkeys (as shown by T in FIG. 1).

3. culturing the elementary neuroepithelial stem cells in the proliferation culture medium to obtain the neuroepithelial stem cells which can maintaine stable proliferation ability.

After culture in the proliferation culture medium, the elementary neuroepithelial stem cells which are obtained by differentiation grow to confluence and afterwards the neuroepithelial stem cells are obtained.

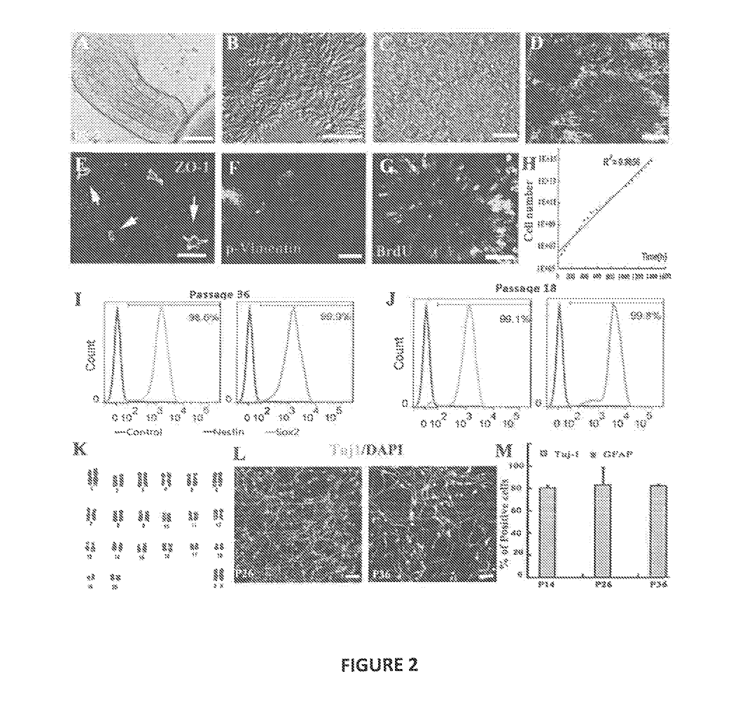

Additionally, in order to enable the cultured cell to self-organize into a neural tube structure, the following operation may be performed: neuroepithelial stem cells need to be continuously cultured on a culture plate for 7-8 days before passaging. These passaged cells maintained exponential growth, rapid growth speed and stable growth abilities even after the 50th passage. By measuring with a fluorescence-activated cell sorter, it is found that these cells stably express the proteins of neural stem cells, for example, Nestin and Sox2, during long term culture. Also, these cells which are passaged and cultured long term keep the property of being differentiated to highly enriched neurons as well as the property of forming the neural tube structure, referring to FIG. 2.

In FIG. 2, it is shown that a neuroepithelial stem cell culture system can be used to culture neuroepithelial stem cells in large scale and for long term, and the stem cells are maintained to self-organize into the neural tube structure and produce highly enriched neurons, wherein (A) shows that an embryoid body of the 6th day, when cultured on a plate pre-coated with laminin, forms a two-layer neuroepithelial structure after 3 days; (B) shows that the neuroepithelial stem cells obtained by differentiation at low cell density condition form a structure of neural rosettes; (C) shows that the neuroepithelial stem cells form the neural tube structure at high cell density; (D-E) show that the neuroepithelial stem cells cultured for long term keep the ability of forming the neutral tubes and expressing Nestin and ZO-1 proteins of the neuroepithelial stem cells; (F-G) show that dividing cells are mainly located on the surfaces of the neural tubes by division protein, phospho-vimentin (p-vimentin), staining, whereas the cells of the DNA synthesis phase (S phase) labeled by BrdU are located on the basal of the neural tubes, illustrating the existence of interkinetic nuclear migration in cells; (H) shows the growth curve of the neuroepithelial stem cells, presenting exponential growth over culture; (I-J) show that measurements with fluorescence-activated cell sorter display the cells of the 18th and 36th passages highly express the similar levels of Sox2 and Nestin; (K) shows that the neuroepithelial stem cells after amplified for 96 generations still have the normal karyotype; and (L-M) show that the Tuj1 neuron differentiation rate is maintained stability and efficiency (>80%) over the long term culture, however the GFAP astrocytes are lacked.

4. digesting the neuroepithelial stem cells with 0.05% trypsin and subjecting them for passaging to obtain a stable neuroepithelial stem cell line.

Preferably, in this step, a ratio of one can use 0.05% trypsin for digestion and passaging at 1:8-1:16. The culture plate is pre-coated with the 5-50 μg/ml laminin for more than 2 hours. Via the above operation of digestion and passaging, a neuroepithelial stem cell line can be obtained and stably passaged.

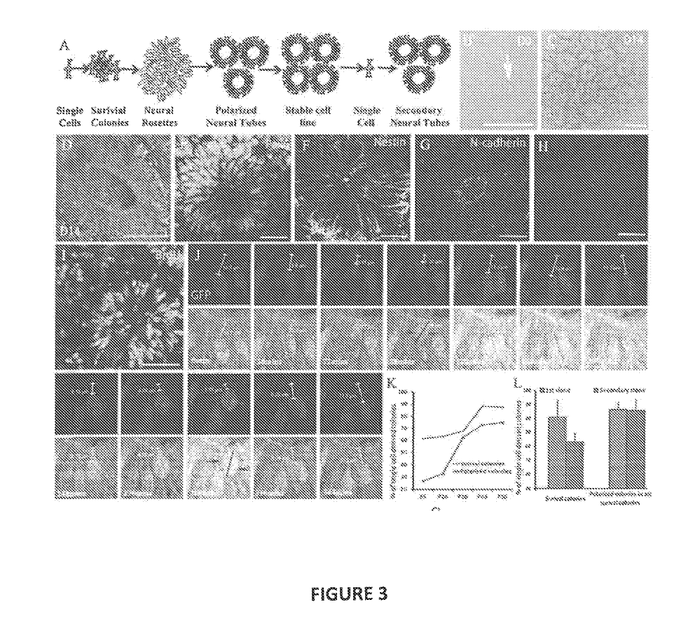

Additionally, a neuroepithelial stem cell can self-organize into the neural tube structures. Preferably, the operation may be performed as follows: neuroepithelial stem cells cultured for long term are digested into single cells, limitedly diluted, and cultured on wells of a 96-well plate coated with laminin, one cell per well, in the above proliferation culture medium; the liquid is changed every 2-3 days, and the single neuroepithelial stem cells self-organize into a series of neural tube structures at the 14-15th day. These neural tube structures derived from single cells express Sox2, Nestin, Pax6, ZO-1 and N-cadherin. The BrdU incorporation shows that these neural tube structures have very strong proliferation ability and display interkinetic nuclear migration during cell division, as shown in FIG. 3.

FIG. 3 shows that single neuroepithelial stem cells self-organize into a neural tube structure. (A) is a continual colony assay of the single neuroepithelial stem cell self-organization into the neural tube structure; (B) shows a neuroepithelial stem cell on the 96-well plate; (C-D) show a typical neural tube colony produced by single neuroepithelial stem cells and (D) shows the single neural tube structure under higher magnification; (E-H) show that the neural tube structure derived from a neuroepithelial stem cell expresses neuroepithelial stem cell proteins, such as Pax6, Nestin, N-cadherin and ZO-1; (I) BrdU incorporation shows that the neural tube structure derived from a cell still has very strong proliferation ability; (J) shows that the neuroepithelial stem cells display interlinetic nuclear migration and horizontally symmetrical division similar to in vivo cells by live imaging; (K) shows percentages of single seeded cells on Day 14 that formed NESCs-derived survival colonies and neural tube colonies at sequential passages, which show that the proportion of the two types of the neuroepithelial stem cell-derived colonies is higher over passaging; (L) shows comparison between the ratio of the survival cell colony Vs. inoculated cells and the ratio of the polarized neural tube Vs. the survival colony during the continuous clonal assays, whose results illustrate no significant difference between the first and secondary colonies (P>0.05).

In addition, in the present embodiment, in order to detect which factors in the whole culture system are essential and necessary for the growth and generation of the neural tubes, it is possible to use the characteristic of the single cell forming the neural tube structure and to perform a series of single cell culture tests where one ingredient is removed from the culture medium every time to estimate which ingredients are essential for self-renewal of the neuroepithelial stem cells and formation of the neural tube.

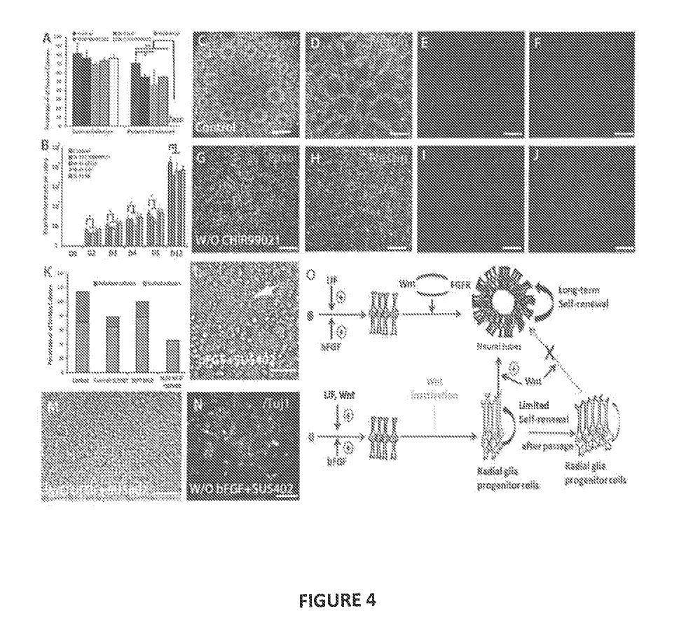

It was found from that removal of SB431542 does not affect the ability of the cells to form neural tubes. Removal of the Leukemia Inhibitory Factor or the bFGF alone results in significant decrease of ability to form neural tubes and of cell proliferation ability. On the contrary, when CHIR99021 was removed, no cell could produce the neural tube structure. When SU5402 is used to inhibit FGFR (Fibroblast growth factor receptor) signaling pathway, all cells lose the ability to produce neural tubes. Thus, the tests demonstrate that the Wnt and FGFR signals are necessary for generating the neural tube structure at the single neuroepithelial stem cell level. Through the secondary clone assays, we found that the Leukemia Inhibitory Factor is necessary for cells to continuously produce neural tube structures. Refer to FIG. 4.

FIG. 4 shows the mechanism by allowing a neuroepithelial stem cell self-organizes into a neural tube structure and is converted to the radial glial progenitor cell. (A) shows comparison of the cell survival ratio and the ratio of the polarized neural tube formation of the single neuroepithelial stem cells under different culture conditions, wherein *P value <0.05 means a significant difference and **P value <0.01 means extremely significant difference; (B) shows a comparison of proliferation differences of single neuroepithelial stem cells cultured at different conditions over the culturing time, wherein a *P value <0.05 means a significant difference; (C-J) show that the activation of the Wnt signaling pathway is necessary for the neuroepithelial stem cells forming neural tubes, and the inactivation thereof would abolish the ability of forming neural tubes and make them conversion to the radial glial progenitor cells; and (K-N) show that the endogenous FGFR signaling pathway is necessary for the formation of neural tubes. SU5402 is an inhibitor of the FGF receptor. What the arrow points to is the miniature neural tube structure derived from the single neuroepithelial stem cell in the neuroepithelial stem cell proliferation culture medium containing SU5402. (O) shows a model of the neuroepithelial stem cell self-organization into the neural tube structure and conversion to the radial glial progenitor cell.

From FIG. 4, the effects of Leukemia Inhibitory Factor, FGF and the Wnt signaling pathway during the self-organization of the neural tube are shown. Exogenous bFGF and Leukemia Inhibitory Factor prompted the self-renewal of the neuroepithelial stem cells and increased the formation of neural tube structures, while endogenous FGFR and Wnt signaling pathway are necessary for the formation of the neural tube. Additionally, when the Wnt signaling pathway is inactivated, the neuroepithelial stem cell is converted to the radial glial progenitor cell and loses the ability to form the neural tube. Moreover, the early-converted radial glial progenitor cells can be re-converted to neuroepithelial stem cells once the Wnt signaling pathway is re-activated, whereas the radial glial progenitor cells are unable to be re-converted to the neuroepithelial stem cells after passage.

After Step 3, optionally, it is also possible to use the neuroepithelial stem cells and single neural stem cells to give rise to highly enriched neurons. Using the stable neuroepithelial stem cell lines produced by the neuroepithelial stem cells and the single cells cultured for long term, under conditions which exclude bFGF, CHIR99021, SB431542 and LIF, spontaneous differentiation occurs, wherein the neuron differentiation culture medium is Neurobasal culture medium supplemented with B27, non-essential amino acids (possible content of up to 1%) and glutamine (possible concentration of up to 1 mM). The differentiated cells undergo the growth phases of the radial glial stem cells (the 0th-3rd day) and the intermediate neural precursor cells (the 4th-6th day), and eventually become highly enriched neurons. These differentiated neurons comprise excitatory glutamergic and inhibitory GABAergic neurons. These neurons also comprise the neurons of the II-IV layer and the V-VI layer of the cortex, and the intermediate neurons of the cortex, as shown by D-J in FIG. 4.

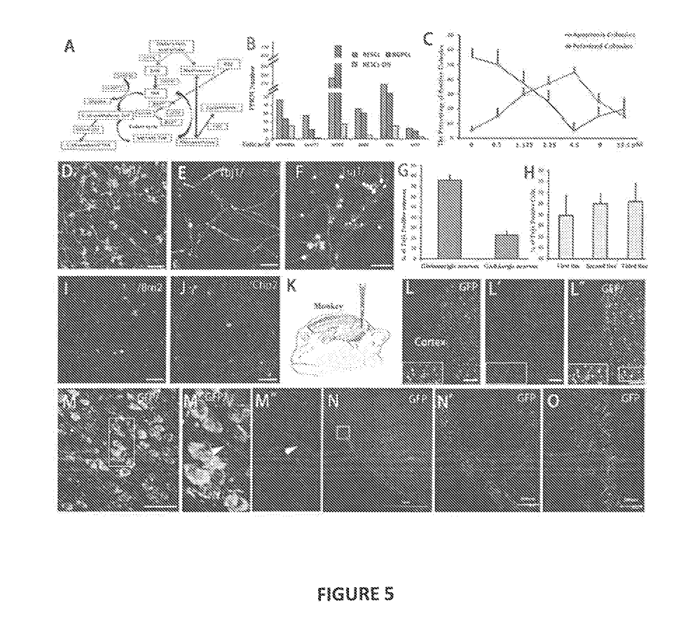

At last, after lentivirus containing GFP are added into the proliferation culture medium for 4 hours, the infected neuroepithelial stem cells are washed off with PBS and the culture medium is replaced with fresh culture medium. The cell line, produced by the single neuroepithelial stem cells labeled with GFP via the above single cell proliferation method, is transplanted to the brain of a monkey under anesthesia by means of a stereotaxic instrument. After three months, the brain is fixed and histology analysis performed. It is found that the transplanted cells integrate into the brain of the monkey and differentiate to neurons. These graft neurons outgrow long axons within 3 months and are widely distributed in the cortex, and some neural axons extend to the deeper layer of the cortex. Therefore, the cells have important clinical value and are capable of being regarded as important donor cell source for stem cell therapy of neural diseases. Refer to L-O in FIG. 5.

FIG. 5 shows a single neuroepithelial stem cell used to model human neural tube disease and give rise to cortical neural cells to perform neural cells replacement therapy. (A-C) show the single neuroepithelial stem cell is capable of modeling the neural tube defect disease by the “single neuroepithelial stem cell to neural tubes” assay, wherein DHFR, SHMT1, MTRR, MTHFD1L, MTR and CBS are key enzymes (A) in folic acid metabolism. These enzymes are highly expressed in the neuroepithelial stem cells (B), and the concentration of the folic acid is in positive correlation with the neural tube formation, and in negative correlation with colony apoptosis (C). (D-J) show that the stable cell line derived from the single neuroepithelial stem cells can be differentiated to cortical neurons. These differentiated neurons comprise excitatory glutamergic and inhibitory GABAergic neurons (D-G). The quantitative results show that there are no significant differences among the proportions of neurons differentiated by three different single cell lines (H). Moreover, these cell lines can differentiate into Brn2 neurons of the II-IV layer and the Ctip2 neurons of the V-VI layer of the cortex, such as I-J. (K-O) show that the neuroepithelial stem cells derived from the single neuroepithelial cells, after being transplanted to the visual cortex of monkeys, can integrate into the cortex (K). These transplanted cells integrate into the outermost layer (L-L″) and the II-IV layer (M-M″) of the cortex, and differentiate to neurons. Also, these differentiated neurons outgrow very long axons, which distribute at multiple point positions of the cortex and extended into the deep layer of the cortex, as shown by O in FIG. 5.

Additionally, the protection scope of the present invention covers the neuroepithelial stem cells created by the method of the present embodiment and the cell line produced by culturing the neuroepithelial cells as well as the applications of the neuroepithelial stem cells and the cell line produced by culturing the neuroepithelial stem cells in the cell replacement therapy, disease mechanism and drug screening.

The above are merely the preferable embodiments of the present invention, and not intended to limit the present invention. As to one skilled in the art, various changes and variations may be made to the present invention. Within the spirit and principle of the present invention, any change, equivalent, improvement and the like should be covered in the protection scope of the present invention.

What is claimed is:

1. A culture medium useful in either inducing differentiation of a pluripotent stem cells to an elementary neuroepithelial cell or proliferation of said elementary neuroepithelial cell, comprising:
(i) a neurobasal culture medium;
(ii) a B-27 supplement;
(iii) an N-2 supplement;
(iv) bFGF;
(v) an agonist of a Wnt signaling pathway;
(vi) a GSK inhibitor;
(vii) an inhibitor of a TGF-β signaling pathway, and
(viii) either both an inhibitor of a Notch signaling pathway and an inhibitor of an ALK2/ALK3 signaling pathway, or leukemia inhibitory factor.

2. The culture medium of claim 1, wherein:
both said agonist of said Wnt signaling pathway and said GSK inhibitor is CHIR 99021, having structure 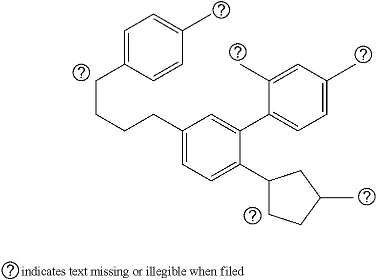 [SMILES: Cc1ccc(CCCCc2ccc(C3CCC(C)C3)c(-c3ccc(C)cc3C)c2)cc1]
said TGF-β signaling pathway inhibitor is SB431542, having structure 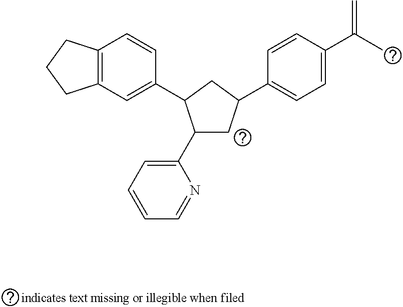 [SMILES: C=C(C)c1ccc(C2CC(c3ccc4c(c3)CCC4)C(c3ccccn3)C2)cc1]
said Notch signaling pathway inhibitor is Compound E having structure of, and 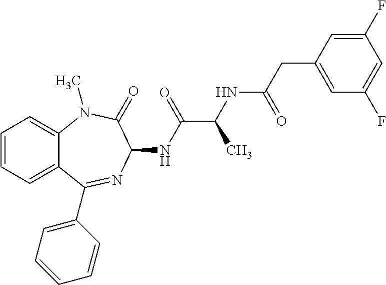 [SMILES: C[C@H](NC(=O)Cc1cc(F)cc(F)c1)C(=O)N[C@H]1N=C(c2ccccc2)c2ccccc2N(C)C1=O]
said ALK2/ALK3 signaling pathway inhibitor is LDN 193189 having structure,

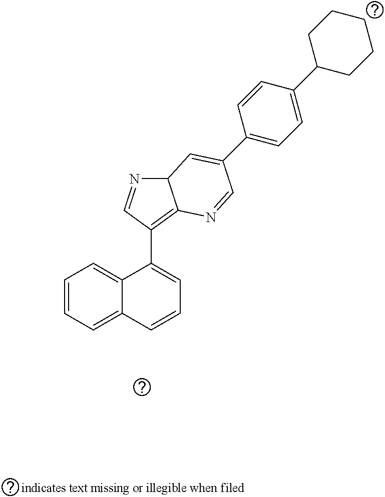 [SMILES: C1=NC2=C(c3cccc4ccccc34)C=NC2C=C1c1ccc(C2CCCCC2)cc1]

3. The medium of claim 2, wherein bFGF is present at a concentration of 3-100 ng/ml, CHIR 99021 is present at a concentration of 0.3-30 |μm/l, SB431542 is present at a concentration of 2-50 μm/l, compound E is present at a concentration of 0.05-10 μm/l, LDN 193189 is present at a concentration of 0.1-1.0 μm/l and LIF is present at a concentration of 50-5000 U/L.

4. A method for obtaining neuroepithelial stem cells, comprising:
(a) digesting a sample of pluripotent stem cells with collagenase;
(b) suspending said cells in the medium of claim 1, wherein (viii) comprises a Notch signaling pathway inhibitor and an inhibitor of ALK2/ALK3 signaling pathway, under conditions favoring differentiation of said cells to elementary neuroepithelial cells;
(c) culturing said elementary neuroepithelial cells in a medium comprising:
(i) A neurobasal culture medium;
(ii) a B-27 supplement;
(iii) an N-2 supplement;
(iv) bFGF;
(v) an agonist of a Wnt signaling pathway;
(vi) a GSK inhibitor;
(vii) an inhibitor of a TGF-p signaling pathway; and
(viii) leukemia inhibitory factor,
under conditions favoring formation of neuroepithelial stem cells capable of stable passage.

5. The method of claim 4, further comprising:
(d) diluting said neuroepithelial stem cells to obtain single cells, and
(e) culturing said single cells in the medium of step (c) for 14-15 days, to obtain neural tubes.

6. The method of claim 4, further comprising culturing said neuroepithelial stem cells capable of safe passaging in a neuron differentiation medium which comprises neurobasal culture medium, B27, non-essential amino acids and glutamine, under conditions favoring differentiation to neurons at a purity of 40-100%.

7. The method of claim 4, comprising digesting said pluripotent stem cells for 5-40 minutes.

8. The method of claim 4, further comprising digesting said neuroepithelial stem cells capable of stable passage with 0.05% trypsin, and passaging said digested cells to obtain a neuroepithelial stem cell line.

9. A neuroepithelial stem cell obtained via the method of claim 4, 5, 6, or 7.

10. A neuroepithelial stem cell line obtained via the method of claim 8.
